(19) United States
(12) Patent Application Publication
(10) Pub. No.: US 2025/0235231 A1
(43) Pub. Date: Jul. 24, 2025

(54) DERMABRASION TOOL WITH SQUEEZEABLE FLUID CONTAINER

(71) Applicant: Gwenaël Bauer, Strasbourg (FR)

(72) Inventor: Torsten Herbert Lippert, Offenburg (DE)

(73) Assignee: Gwenaël Bauer, Strasbourg (FR)

(21) Appl. No.: 19/034,955

(22) Filed: Jan. 23, 2025

(30) Foreign Application Priority Data
Jan. 23, 2024 (DE) 10 2024 101 897.6

(51) Int. Cl. A61B 17/32; A61B 17/00

(52) U.S. Cl. CPC A61B 17/32; A61B 2017/00761; A61B 2017/320004; A61B 2217/007

(57) ABSTRACT

A dermabrasion tool is filled with fluid and has an opening for the fluid to exit. A handpiece is at least partially designed like a container or tube. A dermabrasion area may be arranged or designed in the area of the opening but can also be located at another location in the dermabrasion area.

### CROSS-REFERENCE TO RELATED APPLICATIONS

This application claims priority to German Patent Application No. DE 10 2024 101 897.6 filed Jan. 23, 2024, the entire contents of which are hereby incorporated in full by this reference.

### DESCRIPTION

### Field of the Invention

The invention relates to a dermabrasion tool with a handpiece for manually holding and guiding the dermabrasion tool and a dermabrasion area at the axial end of the dermabrasion tool, wherein the dermabrasion area has a carrier matrix with several friction projections for mechanical skin treatment, which are arranged on the carrier matrix or formed in the carrier matrix, wherein the friction projections each have a maximum cross-sectional width between 0.5 μm and 1000 μm, wherein the handpiece has a cavity filled with a fluid, wherein the dermabrasion tool has an opening fluidically connected to the cavity.

### Background of the Invention

Such a dermabrasion tool is known from EP 3 165 124 B1. The known dermabrasion tool has a dermabrasion area with a friction surface in order to roughen an area of skin. The roughened area of skin can be treated with the fluid.

The disadvantage of the known dermabrasion tool is its rather complex design and the associated high production costs for dosing the respective fluid.

### SUMMARY OF THE INVENTION

### Task of the Invention

It is therefore an object of the invention to provide a dermabrasion tool that can be produced significantly more cost-effectively without giving up the advantages of the known dermabrasion tool, and to simplify the targeted and standardized application of the dermabrasion area together with the fluid.

### Description of the Invention

This object is achieved according to the invention by a dermabrasion tool according to patent claim 1. The dependent patent claims reflect preferred developments.

The object of the invention is thus achieved by a dermabrasion tool as described above, wherein the handpiece is designed to be elastically and/or plastically deformable at least in sections, so that a user can press the fluid out of the opening in portions by pressing and/or turning the handpiece. A user can press the fluid out of the opening simply by pressing and/or turning the elastic and/or plastic section.

The hand part which is deformable at least in sections is also understood to mean a pump part, as is known, for example, from dispensers for liquid soap.

Preferably, the particles have a maximum cross-sectional width between 1 μm and 0.8 mm, in particular, between 1 μm and 350 μm, preferably between 1 μm and 20 μm.

The application surface of the dermabrasion area is preferably round or oval or linear and has a diameter or maximum extension between 1 cm and 5 cm.

The fluid can contain an acid, an alkali, an emulsion/cream and/or a medication. The fluid can contain a pathogen. In this case, penetration via micro-injuries can be used to easily achieve immunizations and/or sensitizations (in allergy sufferers) and/or to administer therapeutics.

The elasticity and/or plasticity of the dermabrasion tool, at least in sections, can be brought about by a handpiece which has a film, in particular, a plastic film.

The handpiece is preferably predominantly elastic, particularly preferably, completely elastic. This makes it particularly easy to press the fluid out of the dermabrasion tool.

The dermabrasion tool can be designed to be particularly simple if the opening is formed in the dermabrasion area. Preferably, the opening is completely surrounded radially by the dermabrasion area or is located next to the opening.

In a particularly preferred embodiment of the invention, the handpiece is designed in the form of a tube or a container. This significantly simplifies the manufacture and use of the dermabrasion tool.

The dermabrasion area can be arranged or formed on a separate component that can be removed from the handpiece. The handpiece including the fluid can thus be replaced by removing the dermabrasion area or the component, so that the dermabrasion area can be applied to a new handpiece. The component can be plugged onto the handpiece or screwed on using a threaded connection. The dermabrasion tool may have a lid to close the opening.

The previously described lid can be designed in the form of a screw lid with a lid thread. The handle can have an axial thread projection with a handle thread matching the lid thread. More preferably, the thread projection can have the opening.

In a further preferred embodiment of this embodiment, the thread projection can have the dermabrasion area.

The removable component may have the threaded projection.

The manufacture of the dermabrasion tool can be further simplified if the carrier matrix is designed in the form of a flat disc.

More preferably, the friction projections can be designed in the form of particles that are embedded in the carrier matrix. The carrier matrix can be made of glass, plastic or paper. The particles can be burned into or applied to the carrier matrix. The particles can preferably be designed in the form of sand grains, diamond dust, glass particles, metal particles and/or plastic particles.

Further advantages of the invention emerge from the description and the drawing. Likewise, the features mentioned above and those described below can be used individually or in combination in any desired way. The embodiments shown and described are not to be understood as an exhaustive list but rather are exemplary in nature for describing the invention.

### BRIEF DESCRIPTION OF THE DRAWINGS

FIG. 1 shows an isometric view of a dermabrasion tool according to the invention.

### DETAILED DESCRIPTION OF EMBODIMENTS OF THE INVENTION

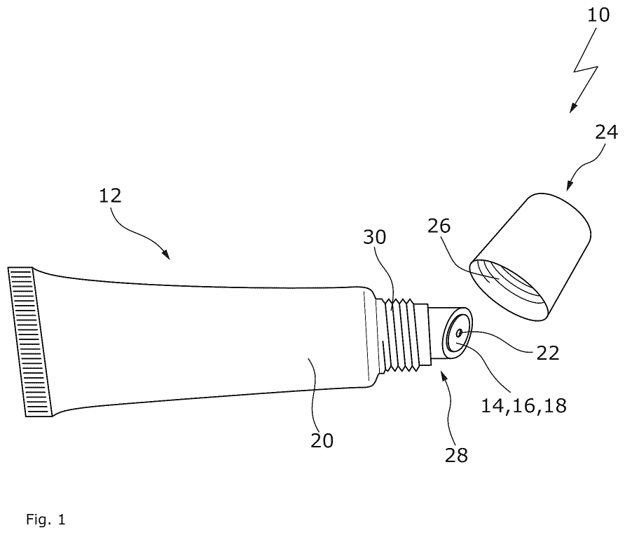

FIG. 1 shows a dermabrasion tool 10 with a handpiece 12 and a dermabrasion area 14, wherein the dermabrasion area 14 has a carrier matrix 16 with several friction projections 18 for mechanical skin treatment.

The handpiece 12 is elastic and/or tube-shaped, so that by pressing, turning and/or pushing the handpiece 12, a fluid 20 inside the handpiece 12 can be pressed out of the handpiece 12 through an opening 22.

The dermabrasion tool 10 has a cover 24 in the form of a screw cap with a cover thread 26. The cover thread 26 is in the form of an internal thread. The handle 12 has a thread projection 28 with a handle thread 30. The handle thread 30 is in the form of an external thread.

The carrier matrix 16 is in the form of a flat disk that is arranged or formed on the front side of the threaded projection 28. In the present case, the carrier matrix 16 surrounds the opening 22.

Looking at the drawing, the invention relates in summary to a dermabrasion tool 10 filled with fluid 20 with an opening 22 for the fluid 20 to exit. A hand part 12 is preferably at least partially designed like a container or tube. A dermabrasion area 14 is preferably arranged or formed in the area of the opening 22, but can also be located at another location in the dermabrasion area 14.

### LIST OF REFERENCE SYMBOLS

10 dermabrasion tools
12 handpiece
14 dermabrasion area
16 carrier matrix
18 friction projections
20 Fluid
22 opening
24 lids
26 cover threads
28 thread projection
30 handpiece threads

What is claimed is:

1. A dermabrasion tool, comprising:
a handpiece configured for manually holding and guiding the dermabrasion tool and a dermabrasion area at the axial end of the dermabrasion tool;
wherein the dermabrasion area has a carrier matrix with several friction projections configured for mechanical skin treatment, which are arranged on the carrier matrix or formed in the carrier matrix;
wherein the friction projections each have a maximum cross-sectional width between 0.5 μm and 1000 μm;
wherein the handpiece has a cavity filled with a fluid;
wherein the dermabrasion tool has an opening fluidically connected to the cavity; and
wherein the handpiece is designed to be elastically and/or plastically deformable at least in sections, wherein a user can press the fluid out of the opening by manually pressing and/or rotating the handpiece.

2. The dermabrasion tool according to claim 1, wherein the handpiece comprises a plastic film wherein it is elastically and/or plastically deformable.

3. The dermabrasion tool according to claim 1, wherein the hand part is predominantly elastic.

4. The dermabrasion tool according to claim 1, wherein the opening is formed in the dermabrasion area.

5. The dermabrasion tool according to claim 1, wherein the hand part is designed in the form of a tube or a container.

6. The dermabrasion tool according to claim 1, wherein the dermabrasion tool has a lid for closing the opening.

7. The dermabrasion tool according to claim 6, wherein the cover is in the form of a screw cap with a cover thread, wherein the handle has an axial thread projection with a handle thread matching the cover thread, wherein the thread projection has the opening.

8. The dermabrasion tool according to claim 7, wherein the threaded projection comprises the dermabrasion region.

9. The dermabrasion tool according to claim 1, wherein the carrier matrix is in the form of a flat disc.

10. The dermabrasion tool according to claim 1, wherein the friction projections are in the form of particles which are embedded in the carrier matrix.
